(19) United States
(12) Patent Application Publication
(10) Pub. No.: US 2005/0002995 A1
(43) Pub. Date: Jan. 6, 2005

(54) Prophylactic article

(75) Inventor: Raimund Schaller, Neunkirchen (AT)

Correspondence Address: GREENBLUM & BERNSTEIN, P.L.C., 1950 ROLAND CLARKE PLACE, RESTON, VA, 20191, US

(73) Assignee: SEMPERIT AKTIENGESELLSCHAFT HOLDING, Wien (AT)

(21) Appl. No.: 10/849,036

(22) Filed: May 20, 2004

(30) Foreign Application Priority Data
May 21, 2003 (AT) A786/2003

(51) Int. Cl. A61K 9/70; A61F 13/00

(52) U.S. Cl. 424/443

(57) ABSTRACT

The invention relates to a multi-layered prophylactic article (1) made from an elastomeric base layer (5) with an internal and an external surface (6, 7), at least a part region of the internal surface (7) having an anti-friction layer (4) made from a polymeric material with an internal surface (9). At least one active substance and/or dye inside particles (11) is disposed on the internal surface (9) of the base layer (5) and/or between the base layer (5) and the anti-friction layer (4) and/or in the anti-friction layer (4) and/or on the internal surface (9) of the anti-friction layer (4), and/or a layer incorporating the at least one active substance and/or dye is disposed in at least a part-region between the base layer (5) and the anti-friction layer (4), which anti-friction layer (4) has regularly recurring raised areas or recesses of an irregular shape, produced by rapidly removing liquid from the anti-friction layer (4).

### CROSS-REFERENCE TO RELATED APPLICATION

The present application claims priority under 35 U.S.C. 119 of Austrian Application No. A 786/2003 filed May 21, 2003, the disclosure of which is incorporated by reference herein in its entirety.

### BACKGROUND OF THE INVENTION

1. Field of the Invention

The invention relates to a multi-layered prophylactic article, in particular a medical glove, comprising an elastomeric base layer, made from a synthetic or natural latex, for example, with an internal and an external surface, at least a part region of the internal surface being provided with an anti-friction layer made from a polymeric material having an internal surface and an external surface facing the internal surface of the base layer, as well as a method of manufacturing it.

2. The Prior art

Medical gloves, whether in the form of gloves used to conduct medical examinations or surgical gloves used for operations, form part of the standard equipment used in medical care. The problem with gloves of this type is that when worn for a longer period, the user's skin can experience irritation or allergic reactions to the various elastomer materials. In order to avoid this, it has been proposed in the prior art that various substances be applied to the skin by means of the glove.

Patent specification U.S. Pat. No. 6,274,154 B1, for example, discloses a therapeutic glove made from a single layer of flexible material and its internal surface has a coating of dehydrated aloe vera. The coating is applied by a process of dipping in a solution containing the aloe vera and the glove is then heated in order to dehydrate and form the aloe vera coating.

Another approach known from the prior art is to micro-encapsulate substances in polyacrylate-polyurethane copolymer dispersions and produce a glove from these dispersions by a dipping process. Utility model DE 201 00 269 U1, for example, describes a coated medical glove with a synergetic anti-HIV action, whereby antiviral substances are enclosed as fillers in spherical individual capsules by a micro-encapsulation technique. The substances become active immediately the capsules rupture under the effect of pressure.

Another glove containing aloe vera oil is known from patent specification U.S. Pat. No. 4,775,372 A and in this case the oil is disposed between two layers of flexible synthetic material in which pouches are formed by spacing the two layers of synthetic material apart from one another. These pouches have to be at least punctured in order to apply the oil, which not only means that certain regions of the glove are not as strong as they could be, but there is also a risk that substances from outside might get into the skin of the user.

### SUMMARY OF THE INVENTION

The objective of the invention is to propose a prophylactic article with improved properties. In particular, the prophylactic article should be such that on the one hand it is easier to put on and on the other hand substances may optionally be released as the article is being put on.

These objectives are achieved by the invention, independently in each case, by means of the aforementioned multi-layered prophylactic article, in which at least a part region on the internal surface of the base layer and/or between the base layer and the anti-friction layer and/or in the anti-friction layer and/or on the internal surface of the anti-friction layer is provided with an active substance and/or dye inside particles, in particular microcapsules, with a maximum diameter selected from within a range with an upper limit of 500 μm, in particular 400 μm, preferably 300 μm, and a lower limit of 10 μm, preferably 30 μm, in particular 40 μm, and/or in which at least a part region between the base layer and the anti-friction layer has a layer containing the at least one active substance and/or dye, and the anti-friction layer has regularly recurring raised areas or recesses of an irregular shape, produced by rapidly removing liquid from the anti-friction layer, of which recesses a proportion selected from a range with a lower limit of 20%, in particular 35%, preferably 40%, and an upper limit of 95%, in particular 80%, preferably 75%, by reference to the total number of recesses, extends through the entire thickness of the anti-friction layer, as well as a method of the type mentioned above enabling the prophylactic article proposed by the invention to be manufactured, whereby the active substance and/or dye is applied by dipping or spraying in particular. The specifically defined surface roughness of the anti-friction layer obtained by the particles and raised areas or recesses has proved to be of advantage not only because it makes the glove easier to put on but also because it simultaneously enables wet slipperiness to be improved. The advantage of this is that, compared with conventional gloves which do not have this wet slipperiness, the active substances and/or dyes are released onto the skin over a longer period and are not totally released due to mechanical stress as the gloves are being pulled on, in particular friction. Another advantage is the fact that the effect of the surface roughness obtained due to the manufacturing process, which makes a prophylactic article easier to put, is further enhanced by the particles. Yet another advantage is the fact that the surface roughness does not produce a full surface contact between the prophylactic article and the human skin, thereby reducing the formation of sweat, and the sweat that is produced can be more readily fed away, thereby making the prophylactic article more comfortable to wear for the user. The reduced surface contact between the prophylactic article and the human skin increases the compatibility of the prophylactic article, reducing incidence of allergic reactions and virtually avoiding them altogether if specific substances are selected. Also of advantage is the fact that the prophylactic article stretches as it is being put on or pulled on, thus reducing the wall thickness, but the other projecting particles reinforce the effect of the surface roughness, also making it easier to put on.

In another embodiment of said invention in which the diameter of the particles is selected from a range with an upper limit of 250 μm, preferably 200 μm, in particular 150 μm and a lower limit of 50 μm, preferably 80 μm, in particular 100 μm, the feeling of touch is totally preserved, especially if the part regions include the terminal phalanges of the fingers.

It is also of advantage if the diameter of the particles is at least 80% of the thickness of the anti-friction layer because this enables the process of manufacturing the prophylactic article proposed by the invention easier insofar as it is not necessary to ensure that the particles within a mixture used to apply the particles are homogeneously distributed, but the surface roughness of the internal surface of the prophylactic article is nevertheless guaranteed, thereby enabling the wet slipperiness to be improved.

The above-mentioned effect is further enhanced if the diameter of the particles is the same as the thickness of the anti-friction coating.

The particles may have a bigger diameter than the thickness of the anti-friction coating, thereby enabling a larger volume of active substances and/or dyes to be incorporated in the particles.

It has also been found to be of advantage if the part region of the prophylactic article includes the region of the distal forearm, the carpal bones, the metacarpals, the base, middle and terminal phalanges of the fingers, in which case the active substances and/or dyes can be dispensed in a perfectly selective manner to the respective body parts, such as the surfaces of the hands, for example, thereby supplying the skin in the respective area with sufficient active substances.

In another embodiment, the particles are applied to both the palm and dorsal areas in at least a part region of the prophylactic articles, which means that not only can the increased moisture requirements of the dorsal side of the hand be catered for, increased perspiration on the palm side can also be avoided.

It is also of advantage if the part region extends on the internal surface of the base layer and/or between the base layer and the anti-friction layer and/or on the internal surface of the anti-friction layer in a range with a lower limit of 40%, preferably 50%, in particular 60%, and an upper limit of 90%, preferably 80%, in particular 70%, which means that the active substance is more reliably released for the wearer of the prophylactic article because of the large surface area to which the active substances and/or dyes are applied.

The particles may be of a different colour from that of the base and anti-friction layers, thereby enabling the distribution of the particles in the prophylactic article to be seen. Another advantage is the fact that rupturing of the particles when subjected to pressure can be visually observed, as a result of which the user of the prophylactic article is assured that the active substances and/or dyes will be released.

Another advantage is the fact that the particles are insoluble in water, as a result of which they can be applied using a dipping process and the active substances or dyes will not be released during the process of manufacturing the prophylactic article, especially during the dipping steps and subsequent washing steps.

In another embodiment, the particles are soluble in water, in which case they may also be applied by means of a spraying process, thereby offering another potential manufacturing process for making the prophylactic article. The fact that the particles release the active substances and/or dyes when they come into contact with the liquid sweat of the user has also been found to be of advantage since this means that there is no need to apply pressure in order to release the active substances and/or dyes, and the active substance will still be released even if the user selects a prophylactic article that is not the correct size.

The active substances may produce an anti-bacterial or anti-viral or antiperspirant or spermicidal or protective effect, thereby enabling many different effects to be achieved by using the prophylactic article. The anti-bacterial and anti-viral effect has proved to be of particular advantage because this prevents infections of a viral and bacterial nature, even if the prophylactic article is torn or damaged. The antiperspirant effect of the active substances reduces the production of sweat by the user, thereby making the prophylactic article more comfortable for the user to wear. The spermicidal effect of the active substances ensures that the contraceptive effect is preserved, for example if a condom is damaged or bursts.

The active substances may be selected from a group consisting of chlorohexidin, e.g. a gluconate, an acetate, a hydrochloride, nonoxinol 9 and aloe vera, thereby imparting an antiseptic and disinfecting action to the prophylactic article so as to prevent a plurality of different bacteria and fungi, as well as certain viruses. These active substances are also substantially harmless and to a certain extent also exhibit protective properties.

The active substances may also be selected from a group consisting of vitamins, plant extracts, in particular secondary plant extracts, thereby providing the prophylactic article with skin care properties in particular.

In another variant of this embodiment, the vitamins may be selected from a group consisting of compounds with a retinoid structure (vitamin A), vitamin B-complex, ascorbic acid (vitamin C), calciferol (vitamin D), tocopherol (vitamin E), vitamin K, flavonoids and biotin, thereby enabling a very individual mix of active substances to be prepared for specific users, depending on requirements.

The concentration of the at least one active substance and/or dye in a particle may be selected from a range with a lower limit of 1%, preferably 2%, in particular 5% and an upper limit of 10%, preferably 15%, in particular 20%, which means that an excessive quantity of active substance and/or dye will not be applied to the skin of the user of the prophylactic article leading to over-supply on the one hand, whilst ensuring a cost-conscious approach to manufacture of the prophylactic article on the other hand.

The fact that the shell of the particles is pressure-sensitive in another embodiment means that at least one active substance and/or dye can be released on exposure to pressure, so that at least some of the active substance and/or dye can be released as the prophylactic article is being pulled on, as a result of which it will be distributed in a substantially homogeneous fashion over the entire hand or body part on which the prophylactic article is being placed as the prophylactic article is pulled over it.

It is of advantage if the particles form the anti-friction layer in the at least one part region, in which case the effect of the surface roughness can be improved, thereby making the prophylactic article easier to pull on.

The thickness of the anti-friction layer may be selected from a range with a lower limit of 30 μm, preferably 40 μm, in particular 50 μm, and an upper limit of 500 μm, preferably 400 μm, in particular 300 μm, and in one variant the range may extend to a lower limit of 55 μm, preferably 60 μm, in particular 75 μm and an upper limit of 200 μm, preferably 150 μm, in particular 110 μm, which will optimise the process of putting on the prophylactic article as well as its wet slipperiness.

In one embodiment, the recesses, as seen in plan view, have a maximum diameter selected from a range with an upper limit of 30 μm, preferably 25 μm, in particular 20 μm, and a lower limit of 1 μm, preferably 5 μm, in particular 10 μm. In yet another embodiment, these recesses may be crater-shaped, tapering in the direction towards the base layer, and in a preferred embodiment the walls of the crater-shaped recesses may subtend an angle relative to the line perpendicular to the anti-friction layer which is selected from a range with a lower limit of 30°, in particular 42°, preferably 47°, and an upper limit of 80°, in particular 75°, preferably 60°. Recesses of this design improve release of the active substance and/or dye, whilst also enabling sweat created during wear to be removed from the skin to the prophylactic article more efficiently, thereby making it more comfortable to wear, and in particular making it easier to remove because it will stick to the skin to a lesser degree.

It is also of advantage if the quantity of active substance and/or dye is selected so that the active substance and/or dye is preferably released in at least more or less equal doses over the entire period during which the prophylactic article is worn, since this will guarantee functionality of the prophylactic article over the whole period it is worn and prevent intermittent excessive release of the active substance and/or dye.

The solubility of the active substance and/or dye in water at 20° C. is preferably selected from a range with a lower limit of 1 g/l, preferably 3 g/l, in particular 4 g/l, and an upper limit of 20 g/l, preferably 15 g/l, in particular 8 g/l, thereby improving the reliability with which the active substance and/or the dye is released and directed onto the skin of the user of the prophylactic article by means of the requisite fluid, in particular sweat.

A solution of the active substance and/or dye in the particles may also have a pH value selected from a range of from 5.5 to 7.5. thereby making the prophylactic article readily compatible with the skin.

In other embodiments, the raised areas are at least substantially in form of a network with inter-connecting webs, a height of at least a part of the webs having a value within the range of between 25% and 100%, preferably 33% and 75%, in particular 40% and 60%, of the total thickness of the anti-friction layer. This reduces direct contact accordingly and hence adhesion of the prophylactic article on the skin, in which case a sufficient number of “pores” is left free so that the active substance and/or dye can be transferred from the interior of the prophylactic article onto the skin.

The particles may be applied in the form of a heterogeneous mixture, in particular a suspension or dispersion, thereby enabling the particles to be processed by a standard method.

At least some of the heterogeneous mixture, in particular the particles, may constitute the anti-friction layer in at least a part region, so that the active substances and/or dyes come into direct contact with the skin surface of the user of the prophylactic article without having to penetrate the anti-friction layer first.

In other embodiments of the invention, the concentration of particles in the heterogeneous mixture may be selected from a range with a lower limit of 1%, in particular 2%, preferably 5% and an upper limit of 50%, preferably 40%, in particular 30% or from a range with a lower limit of 6%, preferably 7%, in particular 10% and an upper limit of 25%, preferably 20%, in particular 15%, so that a sufficient quantity of active substances is made available to the user of the prophylactic article. It has also proved to be of advantage if the concentration is selected so that the prophylactic article can be manufactured cost-effectively.

The method proposed by the invention may be controlled in such a way that the liquid is dispensed within a period with a lower limit of 10 sec., in particular 25 sec., preferably 50 sec., and an upper limit of 20 min., in particular 15 min., preferably 10 min. It has also proved to be of advantage if the liquid is dispensed at a temperature selected from a range with a lower limit of 60° C., in particular 66° C., preferably 70° C., and an upper limit of 150° C., in particular 125° C., preferably 110° C. These embodiments improve the resultant recesses or raised areas, thereby making the prophylactic article made using this method easier to wear, pull on and remove.

Since the other embodiments of the method are used to make the corresponding embodiments of the prophylactic article described above and the features of these claims also be apply to the respective claimed embodiments of the prophylactic article, no further explanations will be given at this stage, in order to avoid repetition, and reference should be made to the explanations given above, including the advantages obtained by the various embodiments, which also apply.

### BRIEF DESCRIPTION OF THE DRAWINGS

To provide a clearer understanding of the invention, it will be explained in more detail with reference to the appended drawings. Of the respective simplified schematic diagrams:

FIG. 1 depicts a prophylactic article in the form of a glove;

FIG. 2 is a cross section through the prophylactic articles along section line II-II indicated in FIG. 1;

FIG. 3 depicts a part of the prophylactic article, seen in section along section line III-III indicated in FIG. 2;

FIG. 4 shows a detail of an embodiment of the prophylactic article illustrated in FIG. 3, viewed in section;

FIG. 5 shows a detail of another embodiment of the prophylactic article illustrated in FIG. 3, viewed in section;

FIG. 6 shows a detail of another embodiment of the prophylactic article illustrated in FIG. 3, viewed in section;

FIG. 7 is an exploded diagram showing a cut-out of an embodiment of the prophylactic articles, in particular a detailed view.

### DETAILED DESCRIPTION OF PREFERRED EMBODIMENTS

Firstly, it should be pointed out that the same parts described in the different embodiments are denoted by the same reference numbers and the same component names and the disclosures made throughout the description can be transposed in terms of meaning to same parts bearing the same reference numbers or same component names. Furthermore, the positions chosen for the purposes of the description, such as top, bottom, side, etc, relate to the drawing specifically being described and can be transposed in terms of meaning to a new position when another position is being described. Individual features or combinations of features from the different embodiments illustrated and described may be construed as independent inventive solutions or solutions proposed by the invention in their own right.

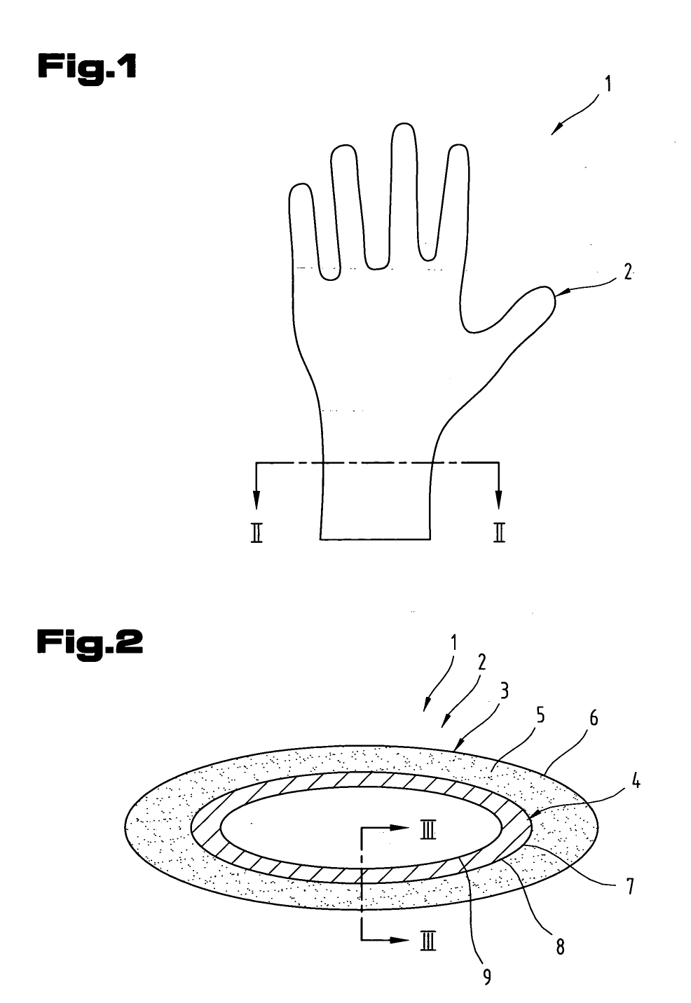

FIGS. 1 to 3 illustrate a prophylactic article 1 proposed by the invention in the form of a glove 2.

In addition to gloves, in particular medical gloves for conducting medical examinations or surgery, the prophylactic article 1 might also be a catheter, condom, finger-stall, bathing cap, flippers or protective gloves for working in clean room environments.

FIG. 2 illustrates a cross section through section line II-II indicated in FIG. 1, depicting the structure of one embodiment of the prophylactic article 1.

The prophylactic article 1 consists of a base material 3 and an anti-friction layer 4 disposed on top of it. The base material 3 forms at least one base layer 5. Naturally, it would also be possible to provide several base layers 5, in which case these base layers 5 could perfectly well fulfil different functions. For example, one of the base layers 5 could be provided in the form of a fabric, thereby improving resistance to any tensile forces which might occur and hence counteracting tearing of the prophylactic article. A fabric layer of this type may be embedded between two base layers 5 covering the entire surface.

The base layer 5 has an external surface 6 and an internal surface 7, the external surface 6 of the base layer 5 being used as the exterior of the prophylactic article 1 and forming an adjacent layer to the internal surface 7 of the immediately adjacent base layer 5. The internal surface 7 of the base layer 5 constitutes either the boundary surface with the external surface 6 of the immediately adjacent base layer 5 or the boundary surface with an external surface 8 of the anti-friction layer 4. The anti-friction layer 4 in turn has an external surface 8 and an internal surface 9, in which case the internal surface 9 of the anti-friction layer 4 faces the skin of the wearer when the prophylactic article 1 is in use.

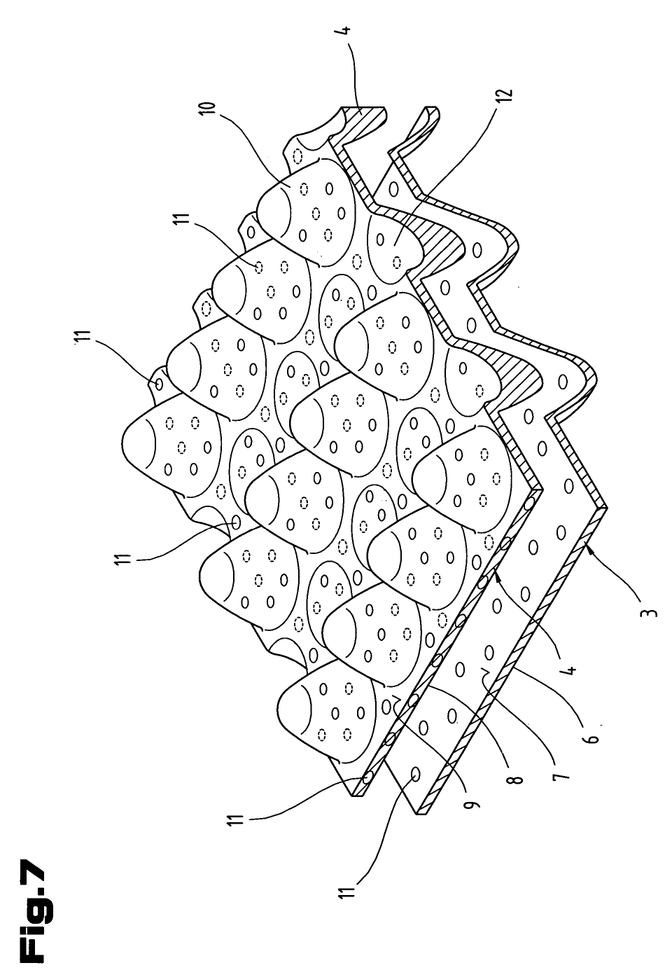

The surfaces 6, 7 of the (individual) base layer(s) 5 and the surfaces 8, 9 of the anti-friction layer 4 may either be smooth, as illustrated in FIG. 2, and if the surface 8, 9 is of a smooth design the surface roughness proposed by the invention is obtained by means of particulate encapsulated active substances and/or dyes, as will be explained in more detail below, or may have structural irregularities 10 (see FIG. 7), in which case a surface roughness is also provided in order to make the prophylactic article 1 easier to put on and improve wet slipperiness. These surfaces with structural irregularities 10 may be made using the manufacturing process described in patent specification EP 0 856 294 B1 or using a method such as that disclosed in patent specification EP 0 856 294 B1.

Multi-layered prophylactic articles 1 made from an elastomeric base material 3, such as gloves 2 for example, are preferably made by dipping a mould of a type known from the prior art into a bath containing the elastomer, in a manner already known from the above-mentioned document. Further explanation is therefore superfluous.

The elastomer may be based on both natural and synthetic latex. In terms of natural and synthetic latex, those which are used by preference are natural rubber, polychloroprene, synthetic polyisoprene, nitrile butadiene and styrene butadiene rubber or a mixture of these polymers. The elastomers used may be non-cross-linked or cross-linked before-hand.

Naturally, all manufacturing methods known from the prior art may be used to produce the base material 3.

The way in which reproducible layers of base material 3 made from elastomers can be made in a mould has long been known to the skilled person and involves applying a coagulant to a mould, for example a ceramic mould with an appropriate roughness or with a smooth surface. To this end, the mould is usually dipped into a tank or container in which the coagulant is placed beforehand in liquid form. This coagulant may be of any composition known from the prior art, such as alcohol solutions of calcium salts or similar, for example. The coagulant may also contain a separating substance such as talcum or calcium carbonate, for example, if it is soluble in acid, which can then be dissolved out of the surface layers during subsequent acid treatments to produce a so-called powder-free glove. The coagulant is then dried.

The mould with the preferably dried coagulant is then dipped into a container in which a supply of elastomer is placed in the form of a dispersion or liquid. In order to increase the layer thickness, the mould can be dipped several times more after briefly drying the latex layer, thereby obtaining an average layer thickness of 100 μm to 300 μm for example. This dipping process may be repeated (several times) if necessary.

The elastomer applied in liquid form hardens due to the chemical reaction of the elastomer with the coagulant. The elastomer is preferably dried for a brief period with hot air immediately after it has been applied to the mould so that the surfaces 6, 7 of the base layer 3 become solid and bonds, and the latter may be fed through an oven or a container in which it is dried with hot air at a temperature of between 70° C. and 140° C. for 15 sec. to 60 sec.

After intermediate drying, the anti-friction layer 4 made from particles 11 containing polymeric material is then applied to the base layer 3 in at least a part region of the surface 7 of the base layer 3, preferably to the entire surface, by dipping or spraying onto the dried base layer 3 in one or more steps. The diameter of the particles 11 may be selected from a range with an upper limit of 500 μm, in particular 400 μm, preferably 300 μm and a lower limit of 10 μm, preferably 30 μm, in particular 40 μm. The diameter of the particles 11 may also have a value selected from a range with an upper limit of 250 μm, preferably 200 μm, in particular 150 μm, and a lower limit of 50 μm, preferably 80 μm, in particular 100 μm.

The layer thickness of the anti-friction layer 4 may be determined on the basis of different requirements, in particular on the basis of the desired roughness depth and the size of the particles 11, and is between 2 μm and 300 μm, preferably 5 μm to 100 μm, in particular 10 μm to 50 μm. The material used for the anti-friction layer 4 may also be applied until the thickness obtained is within a range having a lower limit of 30 μm, preferably 40 μm, in particular 50 μm, and an upper limit of 500 μm, preferably 400 μm, in particular 300 μm, and the anti-friction layer assumes a thickness value selected from a range with a lower limit of 55 μm, preferably 60 μm, in particular 75 μm, and an upper limit of 200 μm, preferably 150 μm, in particular 110 μm. The diameter of the particles 11 is preferably selected so that at least the internal surface roughness of the prophylactic articles 1 is obtained or enhanced. It is of advantage if the particle diameter is at least 80% of the thickness of the anti-friction layer 4 and/or corresponds to the thickness of the anti-friction layer 4. However, it would also be possible for the diameter of the particles 11 to be bigger than the thickness of the anti-friction layer 4.

The anti-friction layer 4 may be made from a heterogeneous mixture of at least one polymer material, such as an aqueous polyurethane dispersion for example, and particles 11, in particular microcapsules, liposomes, etc.

Depending on the intended application, the polymer material might also be a polyacrylate, a polysiloxane, a poly(meth)acrylate, a carboxylated styrene-butadiene copolymer, a polyvinyl pyrolidone, a cationic polyurethane and mixtures thereof, e.g. with a molecular weight of at least 100,000 Da, as well as non-ionic or anionic variants of the materials listed above. The heterogeneous mixture, in particular the aqueous dispersion of the polymeric materials and particles 11 and any mixtures thereof, forms layers or films with good mechanical base properties and preferably has expansion properties similar to those of the base layer 3. The concentration of particles 11 in the heterogeneous mixture may be selected from a range with a lower limit of 1% by weight, in particular 2% by weight, preferably 5% by weight, and an upper limit of 50% by weight, preferably 40% by weight, in particular 30% by weight. The concentration of particles 11 in the heterogeneous mixture is preferably selected from a range with a lower limit of 6% by weight, preferably 7% by weight, in particular 10% by weight and an upper limit of 25% by weight, preferably 20% by weight, in particular 15% by weight.

If the particles 11, in particular the microcapsules, are to be sprayed onto the base material 3, in particular the base layer 5 and/or the anti-friction layer 4, they may have water-soluble properties, so that when the prophylactic article 1 is being worn, the very fact that the particles 11 are in contact with sweat on the user's skin will enable the active substances and/or dyes to be released as the shell material at least partially dissolves.

If the particles 11 are applied to the base layer 5 or anti-friction layer 4 by means of a dipping process, it is preferable if they have water-insoluble properties so that the active substances and/or dyes are not released already during the production process, in particular during the various washing processes. This being the case, it is of advantage if the particles 11 are of the type which rupture under the effect of mechanical stress, such as pressure or friction forces for example, which occur during wearing and in particular when pulling on the prophylactic article 1, thereby releasing the active substances and/or dyes.

The particles 11, in particular microcapsules, contain active substances and/or dyes. The dyes contained in the particles 11 may be of a different colour from that of the anti-friction layer 4 and the base material 3 or base layer 5 in or to which the particles 11 are applied, so that the rupturing of the particles 11 can be visually observed.

The particles 11, in particular microcapsules, may also contain active substances with an antibacterial effect, selected from a group consisting of ammonium iodide, chlorohexidin, chlorohexidin diacetate, chlorohexidin digluconate, chlorohexidin dichloride, hexamidine diisothionate, hexetidine, lauralkonium bromide, aloe vera, lauralkonium chloride, laurtrimonium chloride, lauryl pyridinium chloride, orange skin extract, quaternium 73, benzalkonium chloride, bromochlorophene, 2-bromo-2-nitropropane-1,3-propaniol, captan, cetyl diamonium bromide, cetyl pyridinium chloride, chlorothymol, chloroxylenol, copper PCA, dichlorobenzyl alcohol, nonoxynol-9, etc. The anti-microbial effect of the prophylactic article 1 can be tested using various test methods, such as the agar diffusion method, germ count method, etc., for example.

In an alternative embodiment, the microcapsules contain antiperspirant substances, in particular selected from a group consisting of aluminum glycinate, aluminum chlorohydrate glycinate, aluminum zirconium tetrachlorohydroxyglycine, allantoin derivatives such as alcloxa, aldioxa, aluminum chloride, aluminum chlorohydrex, aluminum PCA, zirconium chlorohydrate and aluminum chlorohydrate, etc., for example.

In an alternative embodiment, the microcapsules contain antiviral and/or germicidal and/or spermicidal substances, all of which are known to the skilled person from specialist literature and thus require no further explanation here.

The particles 11 may also contain protective substances such as glyceryl stearate, glyceryl laurate, octyl stearate, octyl palmitate, tocopheryl nicotinate, PEG, collagen, fruit acids, fatty acids, quercetin, etc., for example.

The microcapsules may also contain vitamins selected from a group consisting of compounds with a retinoid structure (vitamin A), vitamin B-complex, ascorbic acids (vitamin C), calciferols (vitamin D), tocopherols (vitamin E), vitamin K, flavonoids and biotin, trace elements, minerals, plant extracts, in particular secondary plant extracts. It is preferable to encapsulate natural compounds of the vitamins in the particles 11. Naturally, however, synthetic compounds of vitamins may also be used.

Naturally, it would also be possible to apply particles 11 containing substances with different effects to the same prophylactic article 1, in particular in the anti-friction layer 4.

The concentration of the at least one active substance and/or dye in the particles 11 is selected from a range with a lower limit of 1%, preferably 2%, in particular 5%, and an upper limit of 10%, preferably 15%, in particular 20%.

The anti-friction layer 4 may naturally also be made up of a mixture of several differet polymer materials in an aqueous dispersion. This being the case, the dispersion is preferably a mixture of 0% by weight to 30% by weight of polyurethane dispersion, 1% by weight to 40% by weight polyacrylate dispersion, 1% by weight to 20% by weight polysiloxane dispersion and 0% by weight to 10% by weight of fillers, whilst the remaining proportion is water. The fillers which may be used include powder or powdered materials, such as chalk, calcium, gypsum, silicon dioxide and/or maize starch. However, other mixtures may be used to make the anti-friction layer 4 for the prophylactic article 1 proposed by the invention, in particular a glove 2, such as 5% by weight to 15% by weight of polyurethane dispersion, 1% by weight to 8% by weight of polyacrylate dispersion, 2% by weight to 6% by weight of polysiloxane dispersion and 4% by weight to 6% by weight of fillers, the rest being water, as well as in the mixture ratios given below in individual brackets, where the individual proportions as a % by weight of the dispersions of polyurethane, polyacrylate, polysiloxane and fillers are separated by oblique slashes and the remaining quantity up to 100% by weight is made up by adding water. The dry content of the dispersions may be fixed by the skilled person, and the solid content in the case of polyurethane may be between 30% and 50%, in the case of polyacrylate 30% to 50% and in the case of polysiloxane 20% to 40%. These mixtures are as follows (2-7/4-10/3-12/0-5), (0-10/2-6/3-10/3-7), (8-18/5-15/4-7/5-10), (12-22/12-26/16-20/0-4), (17-26/18-32/10-14/2-6), (24-30/28-40/15-20/6-9), (24-30/25-35/5-10/3-7), (21-27/4-9/1-4/6-8), (21-27/12-22/3-9/4-7), (21-27/28-36/12-20/2-7), (9-12/1-3/2-6/5-9), (12-22/4-9/1-4/0-3), (17-26/5-11/7-10/0-4), (2-7/12-22/14-20/3-8), (5-15/28-36/14-20/5-10), (0-10/24-29/9-16/5-8).

At this stage, it should be pointed out for the sake of good order that it would, of course, also be possible for the polymer material of the anti-friction layer 4 and naturally also the elastomer and coagulant solution to be adjusted by adding one or more viscosity regulators of various viscosities known to the skilled person from the prior art in order to adapt to the desired properties. Consequently, the viscosity of the products and materials, usually used in the form of dispersions, can be adapted to suit the intended applications, enabling appropriate layer thicknesses to be obtained during the dipping process or a uniform deposit of the different materials on a surface of the dipping moulds.

The particles 11 may be disposed on at least a part region of the prophylactic article 1 on the palm and/or dorsal side, such as in the region of the distal forearm, the carpal bones, the metacarpals, the base, middle and terminal phalanges of the fingers. Between 40% and 90%, preferably 50% to 80%, in particular between 60% and 70%, of the part regions of the prophylactic article 1 are covered with particles 11, in particular microcapsules.

The specified part regions relate not only to the base layer 3, but also to the anti-friction layer 4.

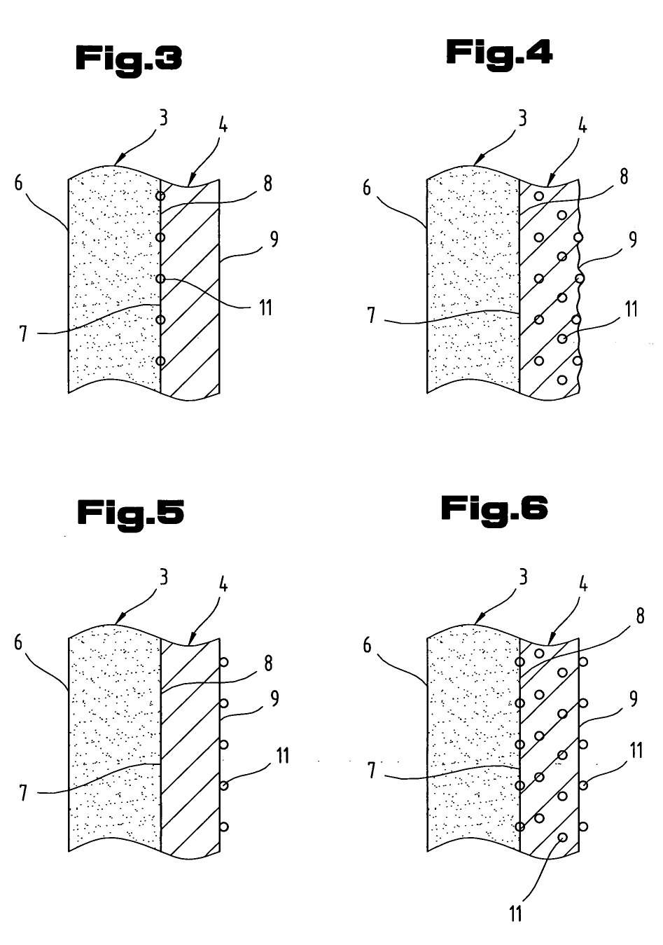

FIGS. 3 to 6 illustrate different embodiments for the arrangement of the particles 11 containing the active substances and/or dyes in the prophylactic article 1.

FIG. 3 illustrates the layout of the particles 11 on the internal surface 7 of the base layer 5 and between the internal surface 7 of the base layer 5 and the external surface 8 of the anti-friction layer 4. The particles 11 may be applied to the internal surface 7 of the base layer 5 by dipping a mould into an aqueous dispersion containing particles 11.

In another embodiment illustrated in FIG. 4, the particles 11 are disposed in the anti-friction layer 4, in which case the particles 11 are applied in a heterogeneous mixture to form the anti-friction layer 4 so that the particles 11 are preferably distributed homogeneously through the anti-friction layer 4.

FIG. 5 illustrates the particles 11 arranged on the internal surface 7 of the anti-friction layer 4, applied as described in connection with FIG. 3.

FIG. 6 illustrates the particles 11 disposed on the internal surface 7 of the base layer 5 and between the internal surface 7 of the base layer 5 and the external surface 8 of the anti-friction layer 4 and in the anti-friction layer 4 and on the internal surface 9 of the anti-friction layer 4, in which case the particles are applied by dipping in dispersion solutions several times.

The different embodiments are intended to show the skilled person that the particles 11 can be disposed in different parts of the prophylactic article 1 to suit requirements, using the technical teaching provided by the invention.

FIG. 7 is an exploded diagram illustrating another embodiment of the prophylactic article 1 and showing a detail, from which it may be seen that the particles 11 may also be arranged on irregularities 10, e.g. raised areas or recesses, of the prophylactic article 1.

After removing the mould carrier containing the dipping mould out of the heterogeneous mixture of polymer material and microcapsules to produce the anti-friction layer 4, the resultant anti-friction layer is dried with hot air, for example at a temperature of between 70° C. and 140° C., preferably 90° C. to 110° C., for a period of 15 sec. to 60 sec. The temperature and duration of the hot air treatment is preferably adjusted so that the surface of the anti-friction layer 4 changes to a gel-like or solid state.

Immediately afterwards, the dipping moulds with the rough parts of the prophylactic article 1 thereon, in particular gloves 2, are dipped in another bath in which the anti-friction layer 4 is sprayed or rinsed with hot water at a temperature of between 40° C. and 95° C., preferably 70° C. to 90° C.

The chemical reaction in the anti-friction layer 4 is initiated or assisted by this hot water treatment, thereby initiating coagulation or fully coagulating this layer.

In the method described above, the roughness and the imparted roughness depth in the anti-friction layer is obtained on the one hand due to the fact that, during and immediately after application of the anti-friction layer 4, between 40% and 70%, preferably 50% to 60%, of the water content of the anti-friction layer 4 is removed with the hot water and on the other hand due to the fact of adding the particles 11 to the anti-friction layer 4. Due to the fact that the water is removed rapidly, a relatively high surface tension is generated in the region of the anti-friction layer 4, which leads to a decrease or reduction in the thickness of the anti-friction layer 4, causing cavities 12 to be created.

The depth of the structural irregularities 10 produced by the method proposed by the invention may be so big that they extend through the entire thickness of the anti-friction layer 4 for example, i.e. as far as the internal surface of the base material 3.

Without departing from the scope of the present invention, it would however also be possible for an anti-friction layer 4 to be provided on both the internal surface 7 and on the external surface 6 of the base material 3, in which case once the base material 3 of the prophylactic article 1 is removed from the dipping mould and placed in the position in which it is intended to be used, in other words with the external surface 6 of the base material 3 lying against the dipping mould, pulled over the same or a different dipping mould, the anti-friction layer 4 can then also be applied to the internal surface 7 of the base material 3, which is then disposed on the dip bath side.

Due to the surface structure resulting from the structural irregularities 10, particles 11 and cavities 12, regions of the anti-friction layer 4 are formed which have a slimmer wall thickness, so that the prophylactic article 1, in particular the glove 2, sits against the surface of the human skin in certain regions only, which thus reduces the contact surface with the skin on the one hand and on the other increases the wet slipperiness in particular. Hollows are simultaneously formed as a result, which are also able to accommodate any sweat which occurs when working, which also significantly improves the comfort with which prophylactic articles 1 of this type can be worn, in particular gloves 2, and because the sweat accumulates to a certain extent, there is enough time for the active substances to be released from the particles 11 before the sweat drains away if the particles 11 have water-soluble shells.

Although the particles 11 are applied to the base layer 5 and/or anti-friction layer 4 by means of dipping or spraying in the examples described above, it would also be possible to use other methods. For example, the surface 7 of the base layer 5 respectively the surface 9 of the anti-friction layer 4 and the particles 11 may be complementarily charged by electrostatic attraction, especially if said surfaces 7, 9, are partially charged so that the particles 11 are selectively directed to and deposited on the described part regions. The particles 11 can be charged by electron bombardment for example or by migration through an electrostatic field.

In another embodiment, not illustrated, the structural irregularities 10, in particular the recesses, may be of an irregular shape, regularly formed on at least a part region of the anti-friction layer 4, in which case this irregular design may be produced in particular by the rapid removal of liquid from the anti-friction layer. This being the case, it has been found to be of advantage if the proportion of recesses extending through the entire thickness of the anti-friction layer 4 is selected from a range with a lower limit of 20%, in particular 35%, preferably 40%, and an upper limit of 95%, in particular 80%, preferably 75%, relative to the total number of recesses in the anti-friction layer 4 of the prophylactic article 1. This will enable crater-shaped recesses to be created, tapering in the direction towards the base layer, preferably with a maximum diameter, as seen in plan view, selected from a range with an upper limit of 30 μm, preferably 25 μm, in particular 20 μm, and a lower limit of 1 μm, preferably 5 μm, in particular 10 μm.

The expression “maximum diameter” used in this connection describes the diameter of a circle enclosing the recesses as seen in plan view.

It should also be pointed out at this stage that the concept of recess or raised area is intended to mean that the material surrounding the recesses may also be seen as a raised area and vice versa, depending on the viewpoint, in other words the expressions recess and raised area in the anti-friction layer 4 may be used differently essentially depending on the reference point.

The rapid removal of water, i.e. liquid, from the anti-friction layer 4, may be achieved as described above or alternatively by another method whereby the liquid is removed, as an alternative or in combination therewith, within a period with a lower limit of 10 sec., in particular 25 sec., preferably 50 sec., and an upper limit of 20 min., in particular 15 min., preferably 10 min. Furthermore, the liquid could be removed, again as an alternative to or in addition, by controlling the temperature, in which case the value of the temperature during the time the liquid is being removed will be in a range with a lower limit of 60° C., in particular 66° C., preferably 70° C., and an upper limit of 150° C., in particular 125° C., preferably 110° C. The requisite temperature may be obtained using conventional means, e.g. various ovens, such as an infrared radiator, for example, or alternatively other heat sources may be used. The hot water method described above may also be used.

By preference, the individual process parameters of the method are combined so that the resultant raised areas are at predominantly of a network type arrangement with inter-connecting webs, and in a preferred embodiment, at least a proportion of the webs with a height corresponding to the total thickness of the anti-friction layer will be in the range of between 25% and 100%, preferably 30% and 75%, in particular 40% and 60%. By using a network-type structure of this design, such as described in the above-mentioned patent specification EP 0 856 294 A1by the same applicant, the contents of which form part of this description, the contact surface between the prophylactic article 1, in particular the anti-friction layer 4, and the user's skin is reduced accordingly, which in turn makes the prophylactic article 1 that much easier to pull on and as a result also easier to pull off. This effect can be further enhanced in the embodiment of the invention described above by using the variants combining particles 11 to produce a certain surface roughness, in which case the particles 11 standing proud of the anti-friction layer 4 will have the effect of making them easier to pull on but, with this combination, the fact of using the particles 11 also means that the release of active substances or dyes to be applied can take place during the pulling-on action, especially if using a water-soluble or pressure-sensitive shell material for the particles 11, which release their contents, in other words the active substances or dyes, making the prophylactic article 1 easier to pull onto the hand, e.g. these active substances and dyes will be at least substantially uniformly distributed over the skin, thereby improving this embodiment over the variant in which the active substances are not released until transported with the fluid through the pores of the anti-friction layer 4 onto the skin, as a result of which the active substances or dyes are distributed on large surfaces of the skin immediately the prophylactic article is pulled on when used for the first time.

To be absolutely clear, it should be pointed out that the expression “pressure-sensitive” used in connection with the shell material of the particles 11 is intended to mean that it bursts when pressure is applied, e.g. during the pulling on process or alternatively due to a corresponding friction force, thereby releasing the active substance and dyes.

It has been found to be of advantage if an active substance and/or dye is used which, at 20° C., has a water solubility selected from a range with a lower limit of 1 g/l, preferably 3 g/l, in particular 4.5 g/l, and an upper limit of 20 g/l, preferably 15 g/l, in particular 8 g/l. Therefore, as an alternative or in combination therewith, the active substance and/or dye is used in a quantity which preferably enables these active substances and/or dyes to be released in at least substantially uniform doses during the time the prophylactic article 1 is being worn, so that release of a partial excessive quantity is avoided relative to time on the one hand, and the active substances and/or dyes are released throughout the entire period they are worn on the other hand.

As regards compatibility with the skin, it has been found to be of advantage to use active substances or dyes which either themselves or made up into a solution have a pH value selected from a range of from 5.5 to 7.5. Preferably, the active substances chosen produce a neutral or slightly acidic medium, although solutions that are slightly basic from a dermatological point of view may also be used without adverse effects.

For the sake of completeness, it should also be pointed out that, as specified in patent specification EP 0 856 294 A, it is preferable to use an elastomer which is already cross-linked or for the most part cross-linked beforehand, although it would naturally also be possible to use elastomers that are not cross-linked. In one embodiment, this elastomer is applied to a dipping mould of a shape corresponding to that of the finished product and coagulated, so as to produce a more or less thin layer of elastic material from the elastomer depending on respective requirements. The dip bath itself contains the usual compounding additives, such as sulphur, zinc oxide, organic accelerators (including zinc salts of dithio-carbamates, thiurams, thioureas, etc., amongst others), stabilisers, waxes, anti-ageing substances, viscosity regulators, fillers, dyes, etc., for example.

For the sake of good order, it should also be pointed out at this stage that the term “elastomer” has been used repeatedly throughout the description, irrespective of whether relating to a dispersion or the cured, dried, cross-linked layer or the solid phase of this material, even if it is usually referred to as rubber, as is the case with natural latex for example. It would therefore also be possible to refer to the layers of individual materials described above as layers of rubber. This polymeric material may be formulated in accordance with the compositions specified above.

The embodiments given as examples illustrate possible variants of the prophylactic article 1 but it should be pointed out that the invention is not restricted to the embodiments specifically described here and instead it would be possible for the individual embodiments to be used in various combinations with one another, in which case these potential variants will be within the reach of the skilled person using the technical teaching of the invention and his technical knowledge of the field. Accordingly, various conceivable embodiments would be possible using combinations of individual details from the embodiments described and illustrated, all of which fall within the scope of the invention even though many of them are not specifically described again.

Finally, for the sake of good order, it should be pointed out that in order to provide a clearer understanding of the structure of the prophylactic article 1, it and its constituent parts are illustrated to a certain extent out of proportion and/or on an enlarged scale and/or on a reduced scale.

The solutions proposed by the invention to achieve the objective may be found in the description. Above all, the individual embodiments of the subject matter illustrated in FIGS. 1, 2, 3; 4; 5; 6; 7 may be construed as independent solutions proposed by the invention in their own right. The objectives and associated solutions may be found in the detailed descriptions of these drawings.

List of Reference Numbers

1 Prophylactic article
2 Glove
3 Base material
4 Anti-friction layer
5 Base layer
6 External surface
7 Internal surface
8 External surface
9 Internal surface
10 Structural irregularity
11 Particles
12 Cavities

1. Multi-layered prophylactic article, in particular a medical glove, made from an elastomeric base layer, such as a synthetic or natural latex for example, with an internal and an external surface, at least a part region of the internal surface being provided with an anti-friction layer made from a polymeric material with an internal surface and an external surface facing the internal surface of the base layer, wherein at least a part region on the internal surface of the base layer and/or between the base layer and the anti-friction layer and/or in the anti-friction layer and/or on the internal surface of the anti-friction layer is provided with at least one active substance and/or dye inside particles, in particular microcapsules, with a maximum diameter selected from a range with an upper limit of 500 μm, in particular 400 μm, preferably 300 μm and a lower limit of 10 μm, preferably 30 μm, in particular 40 μm, and/or a layer incorporating the at least one active substance and/or dye is disposed in at least a part region between the base layer and the anti-friction layer, which anti-friction layer has regularly recurring raised areas or recesses of an irregular shape, produced by rapidly removing liquid from the anti-friction layer, in which a proportion of the recesses selected from a range with a lower limit of 20%, in particular 35%, preferably 40%, and an upper limit of 95%, in particular 80% preferably 75%, by reference to the total number of recesses, extends through the entire thickness of the anti-friction layer.

2. Prophylactic article as claimed in claim 1, wherein the diameter of the particles is selected from a range with an upper limit of 250 μm, preferably 200 μm, in particular 150 μm, and a lower limit of 50 μm, preferably 80 μm, in particular 100 μm.

3. Prophylactic article as claimed in claim 1, wherein the diameter of the particles is at least 80%, preferably at least 85%, in particular at least 90%, of the thickness of the anti-friction layer.

4. Prophylactic article as claimed in claim 3, wherein the diameter of the particles is the same size as the thickness of the anti-friction layer.

5. Prophylactic article as claimed in claim 3, wherein the diameter of the particles is bigger than the thickness of the anti-friction layer.

6. Prophylactic article as claimed in claim 1, wherein the part region encompasses the region of the distal forearm and/or the carpal bones and/or the metacarpals and/or the base, middle and terminal phalanges of the fingers.

7. Prophylactic article as claimed in claim 1, wherein the particles and/or the layer is applied to both the palm side and dorsal side in at least one part region.

8. Prophylactic article as claimed in claim 1, wherein the part region extends across a region of the internal surface of the base layer and/or between the base layer and the anti-friction layer and/or in the anti-friction layer and/or on the internal surface of the anti-friction layer in a range with a lower limit of 40%, preferably 50%, in particular 60%, and an upper limit of 100%, preferably 80%, in particular 70%.

9. Prophylactic article as claimed in claim 1, wherein the particles and/or the layer is a different colour from the base layer and anti-friction layer.

10. Prophylactic article as claimed in claim 1, wherein the particles are water-insoluble.

11. Prophylactic article as claimed in claim 1, wherein the particles are water-soluble.

12. Prophylactic article as claimed in claim 1, wherein the active substance has an antibacterial or antiviral or germicidal or spermicidal or protective action.

13. Prophylactic article as claimed in claim 12, wherein the active substance is selected from a group consisting of chlorohexidin, e.g. a gluconate, an acetate, a hydrochloride, nonoxinol 9 and aloe vera.

14. Prophylactic article as claimed in claim 1, wherein the active substance is selected from a group consisting of vitamins, plant extracts, in particular secondary plant extracts.

15. Prophylactic article as claimed in claim 14, wherein vitamins are selected from a group consisting of compounds with a retinoid structure (vitamin A), vitamin B-complex, ascorbic acid (vitamin C), calciferols (vitamin D), tocopherols (vitamin E), vitamin K, flavonoids and biotin.

16. Prophylactic article as claimed in claim 12, wherein the concentration of the at least one active substance and/or dye in the particles is selected from a range with a lower limit of 1%, preferably 2%, in particular 5%, and an upper limit of 20%, preferably 15%, in particular 10%.

17. Prophylactic article as claimed in claim 1, wherein a shell of the particles is pressure-sensitive.

18. Prophylactic article as claimed in claim 1, wherein the particles form the anti-friction layer in at least a part-region.

19. Prophylactic article as claimed in claim 1 wherein a thickness of the anti-friction layer is selected from a range with a lower limit of 30 μm, preferably 40 μm, in particular 50 μm, and an upper limit of 500 μm, preferably 400 μm, in particular 300 μm.

20. Prophylactic article as claimed in claim 19, wherein the thickness of the anti-friction layer is selected from a range with a lower limit of 55 μm, preferably 60 μm, in particular 75 μm, and an upper limit of 200 μm, preferably 150 μm, in particular 110 μm.

21. Prophylactic article as claimed in claim 1, wherein the recesses have a maximum diameter, as seen in plan view, selected from a range with an upper limit of 30 μm, preferably 25 μm, in particular 20 μm, and a lower limit of 1 μm, preferably 5 μm, in particular 10 μm.

22. Prophylactic article as claimed in claim 1, wherein the recesses are crater-shaped and taper in the direction towards the base layer.

23. Prophylactic article as claimed in claim 22, wherein walls of the crater-shaped recesses subtend an angle with the line perpendicular to the anti-friction layer selected from a range with a lower limit of 30°, in particular 42°, preferably 47°, and an upper limit of 80°, in particular 75°, preferably 60°.

24. Prophylactic article as claimed in claim 1, wherein a quantity of the active substance and/or dye is selected so that the active substance and/or dye is preferably released in at least substantially uniform doses throughout the entire time the prophylactic article is being worn.

25. Prophylactic article as claimed in claim 1, wherein the active substance and/or dye has a solubility in water at 20° C. which is selected from a range with a lower limit of 1 g/l, preferably 3 g/l, in particular 4.5 g/l, and an upper limit of 20 g/l, preferably 15 g/l, in particular 8 g/l.

26. Prophylactic article as claimed in claim 1, wherein a solution of the active substance and/or dye in the particles has a pH value selected from a range of 5.5 to 7.5.

27. Prophylactic article as claimed in claim 1, wherein the raised areas are arranged in an at least predominantly network-type arrangement with inter-connecting webs.

28. Prophylactic article as claimed in claim 27, wherein a height of at least a part of the webs has a value in the range of between 25% and 100%, preferably 33% and 75%, in particular 40% and 60%, of the total thickness of the anti-friction layer.

29. Method of producing a multi-layered prophylactic article, in particular a medical glove, in which at least a base layer is made from an elastomeric material, preferably synthetic or natural rubber, which has an internal surface and an external surface, the internal surface of the base layer having an anti-friction layer of a polymeric material with an internal surface and an external surface facing the internal surface of the base layer, wherein at least one active substance and/or dye inside particles is applied to the internal surface of the base layer and/or between the base layer and the anti-friction layer and/or in the anti-friction layer and/or on the external surface of the anti-friction layer, the particles used having a maximum diameter selected from a range with an upper limit of 500 μm, in particular 400 μm, preferably 300 μm and a lower limit of 10 μm, preferably 30 μm, in particular 40 μm, and /or a layer incorporating at least one active substance and/or dye is applied in the at least one part-region between the base layer and the anti-friction layer, in particular by dipping or spraying, which anti-friction layer has regularly recurring raised areas or recesses of an irregular shape produced by rapidly removing liquid from the anti-friction layer, and a proportion of the recesses selected from a range with a lower limit of 20%, in particular 35%, preferably 40%, and an upper limit of 95%, in particular 80%, preferably 75%, by reference to the total number of recesses, extends through the entire thickness of the anti-friction layer.

30. Method as claimed in claim 29, wherein that the applied particles have a diameter selected from a range with an upper limit of 250 μm, preferably 200 μm, in particular 150 μm, and a lower limit of 50 μm, preferably 80 μm, in particular 100 μm.

31. Method as claimed in claim 29, wherein the particles and/or layer is or are applied in the form of a heterogeneous mixture, in particular a suspension or dispersion.

32. Method as claimed in claim 31, wherein at least a part-region of the anti-friction layer is formed by the heterogeneous mixture.

33. Method as claimed in claim 29, wherein a concentration of particles in the heterogeneous mixture used is selected from a range with a lower limit of 1%, in particular 2%, preferably 5%, and an upper limit of 50%, preferably 40%, in particular 30%.

34. Method as claimed in claim 22, wherein the concentration of the particles in the heterogeneous mixture is selected from a range with a lower limit of 6%, preferably 7%, in particular 10% and an upper limit of 25%, preferably 20%, in particular 15%.

35. Method as claimed in claim 29, wherein the liquid is removed within a period with a lower limit of 10 s, in particular 25 s, preferably 50 s, and an upper limit of 20 min, in particular 15 min, preferably 10 min.

36. Method as claimed in claim 29, wherein the liquid is removed at a temperature selected form a range with a lower limit of 60° C., in particular 66° C., preferably 70° C., and an upper limit of 150° C., in particular 125° C., preferably 110° C.

37. Method as claimed in claim 29, wherein particles with a water-soluble shell are used.

38. Method as claimed in claim 29, wherein particles with a water-insoluble shell are used.

39. Method as claimed in claim 29, wherein the active substance is a substance with an antibacterial or antiviral or germicidal or spermicidal or protective action.

40. Method as claimed in claim 29, wherein the active substance is selected from a group consisting of chlorohexidin, e.g. a gluconate, an acetate, a hydrochloride, nonoxinol 9 and aloe vera.

41. Method as claimed in claim 29, wherein the substance is selected from a group consisting of vitamins, plant extracts, in particular secondary plant extracts.

42. Method as claimed in claim 41, wherein the vitamins are selected from a group consisting of compounds with a retinoid structure (vitamin A), vitamin B-complex, ascorbic acid (vitamin C), calciferols (vitamin D), tocopherols (vitamin E), vitamin K, flavonoids and biotin.

43. Method as claimed in claim 29, wherein the at least one active substance and/or dye is contained in the particles in a concentration selected from a range with a lower limit of 1%, preferably 2%, in particular 5%, and an upper limit of 20%, preferably 15%, in particular 10%.

44. Method as claimed in claim 29, wherein the particles are applied in at least one part-region in the anti-friction layer.

45. Method as claimed in claim 29, wherein the material used for the anti-friction layer is applied until the latter has a thickness selected from a range with a lower limit of 30 μm, preferably 40 μm, in particular 50 μm, and an upper limit of 500 μm, preferably 400 μm, in particular 300 μm.

46. Prophylactic article as claimed in claim 45, wherein the thickness of the anti-friction layer is selected from a range with a lower limit of 55 μm, preferably 60 μm, in particular 75 μm, and an upper limit of 200 μm, preferably 150 μm, in particular 110 μm.

47. Method as claimed in claim 29, wherein the time during which the liquid is removed is selected so that the recesses produced have a maximum diameter, as seen in plan view, selected from a range with an upper limit of 30 μm, preferably 25 μm, in particular 20 μm, and a lower limit of 1 μm, preferably 5 μm, in particular 10 μm.

48. Method as claimed in claim 29, wherein the time during which the liquid is removed is selected so that crater-shaped recesses are formed which taper in the direction towards the base layer.

49. Method as claimed in claim 29, wherein an active substance and/or a dye has a solubility in water at 20° C. selected from a range with a lower limit of 1 g/l, preferably 3 g/l, in particular 4.5 g/l, and an upper limit of 20 g/l, preferably 15 g/l, in particular 8 g/l.

50. Method as claimed in claim 29, wherein a solution of the active substance and/or dye in the particle is adjusted and/or displaced with a buffer so that it has and maintains a pH value selected from a range of 5.5 to 7.5.

51. Method as claimed in claim 29, wherein the raised areas form an at least substantially network-type pattern with inter-connecting webs.

52. Method as claimed in claim 51, wherein the at least a part of the webs are formed with a height having a value in the range of between 25% and 100% preferably 33% and 75%, in particular 40% and 60%, of the total thickness of the anti-friction layer.
